(19) United States
(12) Patent Application Publication
(10) Pub. No.: US 2013/0011384 A1
(43) Pub. Date: Jan. 10, 2013

(54) Use of a Material, Produced from Fungal Fermentation, as a Food Supplement

(76) Inventors: Diego P. Morgavi, Tallende (FR); Hamid Boudra, Clermont-Ferrand (FR)

(21) Appl. No.: 13/636,046

(22) PCT Filed: Mar. 25, 2011
(86) PCT No.: PCT/FR2011/050642

§ 371 Date: Sep. 19, 2012

(30) Foreign Application Priority Data
Mar. 26, 2010 (FR) 1052213

(51) Int. Cl. A61K 36/00; A61P 1/00

(52) U.S. Cl. 424/115

(57) ABSTRACT

The invention relates to the use of a material produced from fermentation of an organic substrate by at least one fungal microorganism belonging to the Monascus genus, for manufacturing a food supplement composition for reducing methane production in ruminants.

### FIELD OF THE INVENTION

The invention relates to the field of food supplements for livestock, specifically ruminants. More specifically, the invention relates to the field of food supplements intended to reduce methane production in ruminants.

### PRIOR ART

The production of methane (CH₄) and carbon dioxide (CO₂) by animals results from the digestion of food ingested by these animals. Methane and carbon dioxide production by animals results from the anaerobic breakdown, by microorganisms present inside the digestive tract, of ingested plant biomass. Ruminants, specifically bovines, ovines, caprines, along with buffalo, deer, and camels, excrete much greater quantities of these gases than monogastric animals. By way of example, it is estimated that a dairy cow produces on average roughly 90 kg of methane annually, whereas a pig produces only 1 kg annually. In ruminants, the produced methane is given off into the atmosphere primarily via the mouth (95% of produced methane) in the form of eructation, and via the lungs after it passes into the blood. A small amount of produced methane (5% of produced methane) is given off via flatulence.

The methane released into the atmosphere by ruminants has been found to represent a loss of approximately 6 to 15 percent of ingested gross energy. Methane production by ruminants has also been found to contribute measurably to increased concentrations of these gases in the atmosphere. It should be borne in mind that methane is currently considered to be one of the gases involved in generating a greenhouse effect resulting in global warming. In terms of both livestock ranching productivity and planetary ecology, it therefore appears advantageous to research means for reducing methane production by livestock ruminants.

Certain studies have shown that increasing the food intake level and the quantity of concentrated food (e.g., energy concentrates) added to ruminants' feed resulted in a reduced proportion of energy lost in the form of methane. However, increasing the quantity of consumed feed necessarily involves increased total methane emission by animals.

The state of the art has shown that adding certain ionophoric antibiotics, such as monensin, to ruminant feed significantly inhibited methane production inside the rumen (Sauer et al., 1998, J. Anim. Sci., Vol. 76: 906-914). However, methanogenic microorganisms are not directly affected and it has been observed that the microbial community present inside the rumen is able to develop resistance to ionophoric antibiotics that, over time, leads to a loss of activity by these antibiotics on methane production by ruminants treated with them (Rumpler et al., 1986, J. Anim. Sci., Vol. 62: 1737-1741).

Using anthraquinone-type compounds in inhibiting methane production, increasing volatile fatty acid production, and increasing feed efficacy has also been described (see U.S. Pat. No. 5,648,258).

Using ionophoric compounds in combination with polycyclic quinone compounds has also been proposed (see U.S. Pat. No. 6,743,440).

Using phthalide compounds that induce increased propionate production and inhibit methane production inside the rumen has also been described (see U.S. Pat. No. 4,333,923). Using heterocyclic trichloromethyl derivatives in order to reduce methane production throughout metabolism in the rumen and to increase propionate production at the expense of acetate, thereby improving the animal's growth rate, has also been described (see U.S. Pat. No. 4,268,510).

Using HMG-CoA reductase inhibitors, such as mevastatin and lovastatin, for reducing methane production inside the rumen has also been described. U.S. Pat. No. 5,985,907 shows the inhibiting effect of mevastatin on the growth of methanogenic archaea. Additionally, research by Miller et al. has shown the inhibiting effect of lovastatin on the growth of methanogenic bacteria (Miller et al., 2001, J. Dairy Sci., Vol. 84: 1445-1448). These authors find that these HMG-CoA reductase inhibitors have the potential to be used as food additives in order to increase animal productivity and to reduce methane production in other methanogenic ecosystems.

Using a protease-resistant bacteriocin, isolated from lactic-acid bacteria, has also been proposed (see European Patent Application No. EP 1,673,983).

Using encapsulated organic acids, specifically fumaric acid, in order to reduce methane production in ruminants has also been described (see PCT Application No. WO 2006/040537).

The effect of adding fats to ruminant feed, which reduces methane production, has also been shown. In particular, it has been shown that fatty acids in feed prevent cellulolytic bacteria, including methanogenic archaea, from attaching to feed particles. According to certain studies, polyunsaturated fatty acids may also have a directly toxic effect on bacterial populations and archaea. This inhibition of bacterial populations is accompanied by an increased percentage of propionic acid within the rumen's contents and reduced methane emissions (Bauchart, 1981, Bull. Tech. CRZV Theix, INRA, Vol. 46: 45-55).

The possibility of performing chemical or biological pretreatment of feed in order to reduce methane production during digestion by ruminants has also been explored. For example, the use of halogenated methane analogues for chemical feed pretreatment has been tested. As a biological feed pretreatment, the implantation of bacteria that are able to carry out reductive acetogenesis at the expense of methanogenesis has also been tested. However, these pretreatment methods involving bacterial implantation lead to undesirable side effects, such as reduced breakdown of plant fibers, the risk of adaptation by implanted exogenous microorganisms, and the possibility of accumulating undesirable residues in meat, milk, or the environment (Demeyer et al., 2000, Ann. Zootech., Vol. 41: 37-38).

Animal food supplements that are prepared from microbial cultures have also been described.

U.S. Patent Application No. 2003030194394 describes the preparation of animal food supplements from microbial cultures containing cholesterol-lowering compounds. Administering these food supplements to livestock is claimed to enable the production of meat and other food products with a lower cholesterol content. U.S. Patent Application No. 20030194394 describes, among other things, the use of Monascus purpureus and Monascus ruber cultures obtained from (i) a substrate composed of glucose, agar, and potato; or (ii) a substrate composed of glucose, peptone, and agar in the preparation of a cholesterol-lowering food supplement. U.S. Patent Application No. 20030194394 does not address the issue of reducing methane production in animals.

The preceding description shows that a broad range of solutions to the issue of reducing methane production in ruminants has been proposed in the state of the art.

However, a need continues to exist in the state of the art for novel means for reducing methane production in ruminants.

### SUMMARY OF THE INVENTION

This invention relates to the use of a product resulting from the fermentation of a substrate by at least one fungal microorganism belonging to the Monascus genus in order to manufacture a food supplement composition intended to reduce methane production in ruminants.

In certain embodiments, the fungal microorganism belonging to the Monascus genus is a fungal microorganism that belongs to the Monascus ruber species.

The invention also relates to a method for reducing methane production in ruminants wherein said ruminants are given an appropriate quantity of a food supplement composition as defined above.

### DESCRIPTION OF THE FIGURES

FIG. 1 illustrates a curve of in vivo methane production by sheep who received feed including a food supplement of the invention for six weeks. The results are expressed as an average of the values obtained for a group of six sheep. Y-axis: in vivo methane production, expressed in liters per day and per animal. X-axis: time, expressed in days. A: period before the feed including the food supplement was given; B: period during which the food supplement was given; C: period after the food supplement was given. The vertical bars represent standard deviation values.

### DETAILED DESCRIPTION OF THE INVENTION

Unexpectedly, we have shown according to the invention that a product of fermentation of a substrate by a microorganism from the Monascus genus is capable, when added as a supplement to the feed of an animal, specifically of a ruminant, of causing a substantial reduction in methane production by this animal, specifically by this ruminant.

Equally unexpectedly, we have shown according to the invention that a product of fermentation of an organic substrate by a microorganism from the Monascus genus does not induce any significant changes in the production of acetate, propionate, or butyrate by said animal, specifically by said ruminant. We have also shown that a product of fermentation of an organic substrate by a microorganism from the Monascus genus does not induce any significant changes in volatile fatty acid (VFA) production.

Hence, the applicant has shown that a product of fermentation of an organic substrate by a microorganism from the Monascus genus, when it is administered to an animal, specifically to a ruminant, as a supplement to its normal feed, reduces methane production by this animal without affecting this animal's ability to metabolize normally the feed that it ingests.

This invention relates to the use of a product originating from fermentation of a substrate by at least one fungal microorganism belonging to the Monascus genus for the manufacture of a food supplement composition intended to reduce methane production in ruminants and other herbivores capable of pregastric fermentation, e.g., camelidae.

In other words, this invention relates to the use of a product originating from fermentation of a substrate by at least one fungal microorganism belonging to the Monascus genus, in a food supplement or as a food supplement, in order to reduce methane production in ruminants.

“Ruminants” include: Addax, Alcelaphus, Alcelaphus buselaphus, Acelaphus caama, Antilocapra americana, Antelope, Heck Aurochs, Musk Oxen, Male Goats, Ibex, Capra, Capra aegagrus, Capra caucasica, Capra Cylindricornis, Capra nubiana, Capra sibirica, Capra walie, Cervoidea, Nanny Goats, Swiss Mountain Goats, Appenzell Goats, Toggenburg Goats, Valais Blackneck Goats, Dwarf Stags, Red Stags, Grant's Gazelles, Thomson's Gazelles, Waller's Gazelles, Gnus, Black Roan Antelopes, Hippotragus, Hippotragus equinus, Hippotragus leucophaeus, Markhors, Grey Dwarf Mouflons, Canadian Bighorn Sheep, Dali Mountain Sheep, Mediterranean Mouflons, Sheep, Okapi, Oryx algazelle, Arabian Oryx, Oryx gazelle, Ovina, Ovis ammon, Ovis orientalis, Pecora, and Urial.

Preferred ruminants include bovines, ovines, and caprines.

Preferred bovines include calves, steers, beef cows, and dairy cows.

Preferred ovines include male sheep and ewes raised for meat as well as ewes raised for their milk.

Preferred caprines includes male goats and nanny goats raised for meat as well as nanny goats raised for their milk.

In general, the fungal microorganism from the Monascus genus is selected from the Monascus albidulus, Monascus argentinensis, Monascus aurantiacus, Monascus barkeri, Monascus bisporus, Monascus eremophilus, Monascus floridanus, Monascus fuliginosus, Monascus fumeus, Monascus kaoliang, Monascus lunisporas, Monascus mucoroides, Monascus olei, Monascus pallens, Monascus paxii, Monascus pilosus, Monascus pubigerus, Monascus purpureus, Monascus ruber, Monascus rubropunctatus, Monascus rutilus, Monascus sanguineus, Monascus serorubescens, and Monascus vitreus species.

In general, the fungal microorganism from the Monascus genus is selected from the Monascus bisporus, Monascus pilosus, Monascus ruber (=Monascus purpureus) species (Samson, R A et al., Introduction to Food and Airborne Fungi, 2004).

In certain preferred embodiments, Monascus belonging to the Monascus ruber species are used. In these embodiments, we preferably use a Monascus strain selected from the group composed of the strains AHU WDCM635 (AHU Culture Collection, Graduate School of Agriculture, Hokkaido University), CCFC WDCM150 (Canadian Collection of Fungal Cultures, Agriculture and Agri-Food Canada), DSMZ WDCM274 (DSMZ—Deutsche Sammlung von Mikroorganismen and Zellkulturen GmbH, DSMZ), IAM WDCM190 (IAM Culture Collection, Institute of Molecular and Cellular Biosciences, The University of Tokyo), JCM WDCM567 (Japan Collection of Microorganisms, RIKEN BioResource Center), MAFF WDCM637 (MAFF Genebank Project, Ministry of Agriculture, Forestry and Fisheries, National Institute of Agrobiological Sciences (NIAS)), UAMH WDCM73 (University of Alberta Microfungus Collection and Herbarium, University of Alberta), ATCC WDCM1 (American Type Culture Collection), CECT WDCM412 (Coleccion Espanola de Cultivos Tipo, Universidad de Valencia), DUM WDCM40 (Delhi University Mycological Herbarium, Department of Botany, University of Delhi), IFO WDCM191 (Institute for Fermentation, Osaka), KCTC WDCM597 (KCTC Korean Collection for Type Cultures, Biological Resource Center, Korea Research Institute of Bioscience and Biotechnology), MUCL WDCM308 (Mycotheque de l'Université catholique de Louvain, Systematic and Applied Mycology Laboratory, Université catholique de Louvain), UPSC WDCM603 (Uppsala University Culture Collection of Fungi, Botanical Museum University of Uppsala), CBS WDCM133 (Centraalbureau voor Schimmelcultures, Fungal and Yeast Collection), CGMCC WDCM550 (China General Microbiological Culture Collection Center, Institute of Microbiology, Chinese Academy of Sciences), FRR WDCM18 (Food Science Australia, Ryde, CSIRO, Food Science Australia), IMI WDCM214 (CABI Genetic Resource Collection, CABI Bioscience UK Centre (Egham)), KUFC WDCM677 (Kasetsart University Fungus Collection, Department of Plant Pathology, Faculty of Agriculture, Kasetsart University), NCPF WDCM184 (National Collection of Pathogenic Fungi, PHLS Mycological Reference Laboratory, Central Public Health Laboratories), URM WDCM604 (Universidade Federal de Pernambuco, Micoteca do Departmento de Micologia), CCF WDCM182 (Culture Collection of Fungi, Department of Botany, Faculty of Science, Charles University, Prague), DAR WDCM365 (Plant Pathology Herbarium, Orange Agricultural Institute), HUT WDCM195 (HUT Culture Collection, Department of Molecular Biotechnology, Graduate School of Advanced Sciences of Matter, Hiroshima University), IOC WDCM720 (Colecao de Culturas de Fungos do Instituto Oswaldo Cruz, Fundacao Oswaldo Cruz), LCP WDCM659 (Fungal Strain Collection, Laboratory of Cryptogamy, Musée National d'Histoire Naturelle), OUT WDCM748 (Department of Biotechnology, Graduate School of Engineering, Osaka University), or VTT WDCM139 (VTT Culture Collection, VTT Technical Research Center of Finland).

In certain preferred embodiments, we use Monascus selected from the Monascus ruber and Monascus purpureus species, as described in the examples. The Monascus ruber strain referenced as DSM 62748 (Deutsche Sammlung von Mikroooganismen, Braunschweig, Germany) may be used, for example.

The various methods for obtaining a product of fermentation by a fungal microorganism from the Monascus genus are known to the expert. In particular, many methods for fermenting organic substrates, including solid organic substrates and liquid substrates, by Monascus have been thoroughly described, including Monascus fermentation methods used for manufacturing relevant compounds, including pigments produced by Monascus.

Preferably, a substrate that includes primarily, essentially, or even exclusively organic substances is used.

The product originating from fermentation of a substrate by Monascus can be obtained by culturing Monascus in a liquid nutrient medium, e.g., using submerged liquid-medium culturing techniques known to the expert.

The product originating from fermentation of a substrate by Monascus can be obtained by culturing Monascus in a solid nutrient medium, using techniques known to the expert.

The product originating from fermentation of a substrate by Monascus can be obtained by culturing Monascus in a solid/liquid system, using techniques known to the expert.

In certain embodiments, the product originating from fermentation of a substrate by Monascus is obtained by culturing Monascus on steamed rice, on bread, on bran, on grains, or on grain-based substances, including grain-based foods.

In certain embodiments, said substrate may consist of any type of nutrient medium adapted for culturing fungal microorganisms known to the expert. In other embodiments, the product originating from fermentation of an organic substrate by Monascus is obtained by culturing Monascus in a nutrient medium containing maltitol, as described in French Patent Application No. FR 2,505,856. The culturing of Monascus in a nutrient medium containing maltitol can be performed (i) in a culture submerged in a liquid medium or (ii) in a solid-medium culture.

In still other embodiments, the product originating from fermentation of an organic substrate by Monascus is obtained by culturing Monascus on cellulose, including bacterial-origin cellulose, as is described, e.g., by Sheu et al. (2000, Journal of Food Science, Vol. 65(2): 342-345).

In yet other embodiments, the product originating from fermentation of an organic substrate by Monascus can be obtained by using any of the Monascus culturing methods described in the publications of Yuan-Kun et al. (1995, Journal of Fermentation and Bioengineering, Vol. 79(5): 516-518, Pastrana et al. (1996, Acta Biotechnologica, Vol, 16(4): 315-319), Ahn et al. (2008, Biotechnology Progress, Vol. 22(1): 338-340), or Zhou et al. (2009, Vol. 228(6): 895-901).

As was stated above, the Applicant has shown that a substrate adapted for the preparation of the fermentation product originating from Monascus can be obtained from grains, specifically from grains belonging to the Triticum genus and to the Oriza genus. Substrates prepared from these plant sources provide both the compounds needed for culturing Monascus and those needed for production by Monascus of metabolites that limit methane production by methanogenic bacteria within the ruminal fluid.

Hence, in certain embodiments, the product originating from fermentation is obtained by culturing Monascus on a solid substrate prepared from one or several products selected from the group composed of grains belonging to the Triticum genus, grains belonging to the Oriza genus, and products derived from said grains.

The grains belonging to the Triticum genus include but are not limited to hard wheat (Triticum turgidum), common wheat (Triticum aestivum), einkorn wheat (Triticum monococcum), spelt (Triticum spelta), and triticale (Triticum secale).

The grains belonging to the Oriza genus include the various species of rice. Any part of the grain plant may be used. Nevertheless, cereal grains and seeds are preferably used. The grains and seeds may be whole grains—that is, not hulled—or grains whose bran and, if applicable, germ have been removed. The cereal grains used for preparing the fermentation substrate may come from a sole plant species or may be composed of a mixture of grains from several plant species.

Preparation of the fermentation substrate generally includes a sterilization Step for the cereal grains and seeds. This sterilization step eliminates microbial species present on the grains that might interfere with the development of Monascus.

Hence, in a specific embodiment, the fermentation substrate is a solid, sterilized substrate prepared from cereal seeds.

Preparation of the fermentation substrate may include several steps that preferably occur prior to the optional sterilization step.

The cereal seeds may be quickly ground or crushed, macerated in an appropriate liquid such as water, or precooked. These optional steps aim to make the seeds' nutrient reserves accessible and in a form that is well-suited to fermentation by Monascus.

The method for preparing the fermentation substrate depends upon the cereal seeds used. By way of example, if the substrate is prepared from whole grains such as whole wheat, it is preferable to crush the seeds. The expert's general knowledge on the topic will enable him/her to determine the appropriate methods for preparing the fermentation substrate.

As was illustrated in the examples of this description, the method for preparing the fermentation substrate from grains generally includes a maceration step in an appropriate liquid, preferably water, for several hours prior to the sterilization step. This maceration step makes it possible to set the dry matter content at approximately 50 to 60% by weight of the total weight of the substrate.

The Applicant has shown that adding bran, such as wheat bran, to the fermentation substrate may encourage the growth of Monascus. Hence, in certain embodiments, the product originating from fermentation of a substrate by Monascus is obtained by culturing Monascus on a substrate prepared from cereal seeds and bran, preferably wheat bran.

In this embodiment, the wheat bran may be added to the substrate before or after the sterilization step, and even simultaneously during inoculation of the substrate by Monascus.

In general, during preparation of the fermentation substrate, it is possible to add known nutrient compounds to the cereal seeds in order to encourage the development of Monascus. Nevertheless, the Applicant has shown that this addition of nutrient compounds is not a necessary condition for the development of Monascus nor for the production of fermentation metabolites that are able to act upon methane production by methanogenic bacteria within the ruminal fluid.

Therefore, in certain embodiments, the fermentation substrate is prepared from cereal seeds, to which bran may optionally be added, in the absence of any additional nutrient compound.

In other words, a solid fermentation substrate for preparing the food supplement of the invention does not include nutrient compounds that are extrinsic to the cereal seeds and bran.

By “nutrient compounds,” we mean known compounds that constitute carbon or nitrogen sources and that are generally used for culturing yeasts and molds. As examples of nutrient compounds, we may mention sugars such as sucrose, glucose, maltose, maltitol, sorbitol, and mannitol, and nitrogenous organic molecules such as amino acids, peptides, and peptones.

In certain embodiments, the fermentation substrate consists of a solid substrate obtained by crushing, macerating, and sterilizing cereal seeds belonging to the Triticum genus.

The fermentation product of Monascus can be incorporated in various forms into the food supplement intended to reduce methane production in ruminants.

Hence, in certain embodiments, the product of culturing Monascus on a substrate is used as-is as a food supplement composition intended to reduce methane production in ruminants.

In other embodiments, the product of culturing Monascus on a substrate is used as-is, in combination with one or several other dietarily-acceptable compounds, as a component included in a food supplement composition or a feed ration intended to reduce methane production in ruminants.

In still other embodiments, the product of culturing Monascus on an organic substrate undergoes one or several extraction or refining steps, then the extracted or refined product is used alone or in combination with one or several other dietarily-acceptable compounds as a food supplement composition intended to reduce methane production in ruminants.

Therefore, in certain embodiments, the product of culturing Monascus on an organic substrate undergoes one or several steps involving extraction using solvents, preferably organic solvents; then, the extract is dried in order to provide a dry extract composition that can be used as-is as a food supplement, or said extract composition is combined with one or several dietarily-acceptable compounds in order to obtain said food supplement.

In preferred embodiments, the product of culturing Monascus on an organic substrate undergoes one or several steps involving extraction using ethanol, then the ethanolic extract is dried in order to provide a dry extract composition that can be used as-is as a food supplement, or said dry ethanolic extract is combined with one or several dietarily-acceptable compounds in order to obtain said food supplement.

Hence, in certain embodiments, the use described above is characterized in that the product originating from fermentation of an organic substrate by at least one fungal microorganism belonging to the Monascus genus consists of an extract of a product of fermentation by Monascus involving one or several organic solvents.

In certain embodiments, said extract consists of an ethanolic extract. An ethanolic extract may be obtained from the organic substrate fermented by a Monascus according to ethanol extraction techniques known to the expert. One may, for example, use an ethanol solution having 50% to 100% ethanol by weight in relation to the total weight of the extraction solution. Therefore, an extraction solution may be used that has at least 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% ethanol by weight in relation to the total weight of the extraction solution.

In actual practice, an appropriate volume of ethanolic extraction solution is added to the product fermented by Monascus and the solid/liquid mixture is homogenized, e.g., in a step involving exposure to an ultrasound source of appropriate power for a time period ranging from 15 minutes to 2 hours, preferably for 2 hours. Next, the extraction liquid is separated from the solid particles, e.g., by centrifuging, and the extraction liquid is retained. Ethanolic extraction may be repeated on the solid material resulting from this separation. Thus, 1 to 5 ethanolic extraction steps are performed as described above, preferably two ethanolic extraction steps, then the liquid extraction fractions are added together and preferably filtered in order to eliminate the solid particles still in suspension. Next, the ethanolic extracts are kept, e.g., at 4° C. away from light, or the ethanol is evaporated, e.g., using a Rotavapor®-type device. In other embodiments, the liquid ethanolic extract is freeze-dried.

By way of illustration, for ethanolic extraction of rice fermented by Monascus, 50 ml of freshly-prepared 75% (v/v) ethanol is added to 20 g of fermented rice. After the culture is dilacerated, extraction is performed using ultrasound for 60 min. Extraction is repeated a second time. The 2 extracts are then filtered and kept at 4° C. away from light until they are analyzed.

Unexpectedly, we have shown in the examples that a product of in vitro fermentation of an organic substrate by at least one strain of Monascus significantly reduces methane production, e.g., by over 90%, whereas under the same conditions monacolin K, which was described in the state of the art as a methane production inhibitor, induced no methane-production-reducing effect. As a result, in a food supplement used according to the invention, the effects of the product resulting from fermentation by Monascus cannot be attributed solely to the possible presence of monacolin K in the composition.

According to one advantageous aspect, it should be noted that Monascus species are entirely harmless to humans and animals. Fungi from the Monascus genus have been used in China for two thousand years in human foodstuffs and in human medicine. Specifically, Monascus species are rated as “QPS” (that is, “Qualified Presumption of Safety”) by the European Food Safety Authority (EFSA). Additionally, Monascus species are rated as “GRAS” (that is, “Generally Recognized as Safe”) by the U.S. Food and Drug Administration (FDA).

We have shown in the examples that a major reduction in methane production is obtained with the fermentation products obtained using Monascus strains.

We have shown in the examples that a major reduction in methane production is obtained both (i) with extracts obtained from products of fermentation by Monascus and (ii) with the raw fermentation product that has not undergone any further treatment, e.g., a fermentation product of steamed rice that has not undergone any further extraction operation.

We have also shown that a general reduction of in vitro gas production, as well as a reduction in the production of volatile fatty acids (VFA) both in vitro and in vivo, are obtained with a product of fermentation of a substrate by Monascus spp.

Moreover, we have shown in the examples that supplying ruminants with a food supplement of the invention based on a product originating from fermentation of an organic substrate by at least one fungal microorganism belonging to the Monascus genus causes a reduction of approximately 30 percent of methane production by these ruminants. This effect of significantly reducing methane production has been shown by using, as a food supplement, the primary product of fermentation by Monascus on a substrate composed of steamed rice.

In ruminants that have received a food supplement in accordance with the invention, we have observed an increase in fermentation-related production of propionate inside the rumen, at the expense of acetate production.

We have also shown that, in animals that have received a food supplement of the invention, a major reduction in the number of methanogenic Archaebacteria is created, while at the same time no change is observed in the number of other bacterial organisms or protozoal organisms.

Therefore, it follows from the example results that a food supplement in accordance with the invention reduces methane production in animals, including ruminants, and simultaneously increases ruminal fermentation of feed provided to these animals.

In a food supplement composition used in accordance with the invention, the product originating from fermentation of an organic substrate by at least one fungal microorganism belonging to the Monascus genus, e.g., a dry ethanolic extract, is present in the amount of 0.1% to 100% by weight in relation to the weight of dry matter of said composition. Consequently, a food supplement composition used in accordance with the invention includes 0% to 99.9% by weight of one or several dietarily-acceptable compounds, in relation to the weight of dry matter of said composition.

By a “dietarily-acceptable” compound, we mean any type of compound that is allowed under administrative regulations concerning animal feed, specifically feed intended for livestock ruminants, including bovines, ovines, and caprines.

Dietarily-acceptable compounds include food preservatives, food dyes, sweeteners, flavor enhancers, pH regulators including acidifiers, antioxidants, and texturizing agents.

Dietarily-acceptable compounds include compounds that are likely to be metabolized by the organism, including vitamins or vitamin precursor compounds, carbohydrate compounds such as sugars, fats, and mineral salts.

Dietarily-acceptable compounds also include compounds that are not metabolized by the organism, such as fillers, e.g., natural or synthetic edible polymers, including xanthan gum and algae extracts.

Dietarily-acceptable compounds include food additives defined (i) by European Union Directive 89/107/CEE, issued on Sep. 18, 1989, which lists the categories in its appendix and (ii) by Directive 95/2/CE relating to food additives other than dyes and sweeteners. The various food additive categories include the following categories: Acidifier, Firming Agent, Coating, Filler, Wheat Processing Agent, Modified Starch, Foaming Agent, Anti-caking Agent, Anti-foaming Agent, Antioxidant, Dye, Preservative, Acidity Corrector, Sweetener, Emulsifier, Enzyme, Thickener, Flavor Enhancer, Gelling Agent, Moistening Agent, Leavening Powder (or Leavening Agent), Emulsifying Salt, Sequestering Agent, Stabilizer, Support.

This invention also relates to a method for reducing methane production in ruminants, wherein said ruminants are supplied with an appropriate quantity of a food supplement composition as defined above.

This invention additionally relates to a food supplement composition for reducing methane production in ruminants that includes a product originating from fermentation of a substrate by at least one fungal microorganism belonging to the Monascus genus.

In general, the above food supplement composition is provided to the ruminant in the form of successive intakes spread out over time, e.g., once a day, twice a week, once a week, or twice a month.

Preferably, the ruminants are supplied with the appropriate quantity of the above food supplement composition in a once-a-day dose.

For ruminants who feed exclusively by grazing, it is obvious that the above food supplement composition is supplied separately from their main feed source.

For ruminants that are fed fresh fodder or stored fodder (e.g., hay), or commercially-available food compositions, including food concentrates, the food supplement composition of the invention may be supplied (i) either mixed with said feed, or (ii) in a form that is separate from said feed.

In general, when the food supplement composition as defined in this description takes the form of a dry extract, e.g., a dry ethanolic extract, the daily quantity given to the ruminants is approximately 1 to 100 grams of food supplement composition per kilogram of feed consumed by (or given to) the animal. A daily quantity of at least 1 gram of said food supplement composition includes a quantity of at least 2, 3, 4, 5, 6, 7, 8, 9, 10, 11, 12, 13, 14, 15, 16, 17, 18, 19, or 20 grams of said food supplement composition. A quantity of 100 grams at the most of said food supplement composition includes a quantity of, at the most, 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51 grams of said food supplement composition.

This invention is also illustrated by the following examples.

### EXAMPLES

### Example 1

### Manufacturing Protocol of a Food Supplement Composition (Primary Product of Fermenting Monascus on Steamed Rice)

Tap water is added to nonstick rice (e.g., nonstick rice sold under the brand names Uncle Ben's® or Lustucru®) and allowed to macerate overnight at the temperature of +4° C. Next, the excess water is eliminated, e.g., by passing the solid/liquid macerated composition through a sieve with an appropriately-sized mesh.

The macerated rice is sterilized by placing it inside an autoclave at 121° C. for 15 min. After it is cooled to ambient temperature (20° C. to 25° C.), the macerated and sterilized rice is inoculated with a piece of gelose from a Monascus ruber culture and the material is homogenized so that the Monascus ruber spores are thoroughly distributed throughout the mass of macerated and sterilized rice.

The mixture of rice and Monascus ruber is then incubated under aerobic conditions at the temperature of 30° C. and in darkness for a period of 2 to 3 weeks. The mixture undergoing in vitro aerobic fermentation is stirred daily during the first 3 days of incubation.

The mixture obtained at the end of the two- to three-week period of aerobic fermentation may be used as-is as a food supplement, or it may be dried prior to use.

### Example 2

### In Vitro Effects of a Food Supplement Composition on Metabolite Production by Methanogenic Archaea

### A. Material and Methods

The effect of a rice extract fermented by a selected strain of Monascus sp., prepared in accordance with Example 1, on ruminal fermentation, specifically on methane production, was studied in vitro by using a sequential batch fermentation system.

Three sheep equipped with rumen cannulae, receiving hay-based feed twice a day, were used as ruminal content donors. The ruminal contents of the three animals were sampled in the morning before feeding, filtered through a cloth with a mesh size of 400 μm in diameter in order to obtain the liquid phase, and mixed in equal quantities. To seventy-five milliliters of this mixed ruminal fluid was added 300 ml of anaerobic buffer solution (Weller & Pilgrim, Br. J. Nutr. 32:341-51. 1974). An aliquot fraction (5 ml) of this ruminal fluid/buffer solution was then transferred into Hungate tubes in a CO₂ atmosphere containing 100±2 mg of alfalfa hay. To each tube was added, respectively: (i) 100 μl of the Monascus extract, or (ii) pure monacolin K, (iii) a 25% (v/v) ethanol solution, or (iv) water (see Table 1 below). The tubes were incubated at 39° C. for 48 hrs. Each treatment was performed three times.

TABLE 1

| | Solutions |
|---|---|
| Treatments | (100 μl/tube) |
| 1. Monascus extract | 1 mg/ml eq. monacolin |
| 2. Monacolin K | 1 mg/ml monacolin^{¶} |
| 3. Ethanol control | 25% ethanol |
| 4. Water control | ddH₂0 |

^{¶}The final concentration of monacolin K inside the fermentation tubes is 20 μg/ml

After 48 hrs. of incubation, 2 ml of each of the triplicates of each treatment were mixed, and 1.5 ml of this mixture was used to inoculate 3 new Hungate tubes containing 3.5 ml of buffer and 100 mg of alfalfa hay.

Next, 100 μl of the various treatments was added to the tubes (Cf. Table 1 above), and the tubes were again incubated at 39° C. while stirring for an additional 48 hrs. This procedure was repeated a second time.

At the end of each 48-hr. period, gas production and concentrations of methane and of volatile fatty acids resulting from fermentation were measured.

### B. Results

The results are presented in Table 2 below.

TABLE 2

| | | | | | |
|---|---|---|---|---|---|
| Gas | Methane | | Acetate | Propionate | Butyrate |
| (ml) | μmol | Total VFA | (mmol · l⁻¹) | (mmol · l⁻¹) | (mmol · l⁻¹) |

1^{st} Transfer

| | | | | | | |
|---|---|---|---|---|---|---|
| Monascus 7 Extract | 21.5 ± 1.6 | 162.0 ± 7.7 | 106.5 ± 8.6 | 76.6 ± 5.7 | 19.3 ± 2.6 | 7.5 ± 0.4* |
| Monacolin K at 20 μg/ml | 21.9 ± 0.3 | 162.5 ± 22.9 | 96.3 ± 2.1 | 69.6 ± 1.5 | 16.7 ± 0.4 | 6.1 ± 0.1 |
| Ethanol control | 22.0 ± 0.2 | 155.9 ± 4.5 | 98.3 ± 1.4 | 71.0 ± 0.5 | 17.0 ± 0.5 | 6.6 ± 0.3 |
| Water control | 27.1 ± 0.1 | 132.0 ± 23.3 | 90.0 ± 1.2 | 60.3 ± 0.8 | 18.5 ± 0.3 | 6.6 ± 0.1 |

2^{nd} Transfer

| | | | | | | |
|---|---|---|---|---|---|---|
| Monascus 7 Extract | 16.8 ± 0.4* | 24.6 ± 37.0 | 77.8 ± 5.5 | 48.3 ± 3.6 | 23.3 ± 1.1 | 5.0 ± 0.7 |
| Monacolin K at 20 μg/ml | 17.8 ± 0.5 | 16.2 ± 17.0 | 76.5 ± 7.4 | 49.1 ± 3.8 | 19.7 ± 2.8 | 5.9 ± 0.7 |
| Ethanol control | 18.8 ± 1.7 | 57.8 ± 48.6 | 86.3 ± 17.2 | 58.9 ± 12.3 | 19.1 ± 3.3 | 6.1 ± 1.0 |
| Water control | 19.9 ± 0.0 | 79.3 ± 14.0 | 95.0 ± 1.1 | 60.8 ± 1.1 | 23.5 ± 0.2 | 6.5 ± 0.2 |

3^{rd} Transfer

| | | | | | | |
|---|---|---|---|---|---|---|
| Monascus 7 Extract | 18.2 ± 0.1* | 12.8 ± 10.4* | 90.6 ± 1.6 | 50.8 ± 1.0* | 32.1 ± 0.5* | 5.4 ± 0.2* |
| Monacolin K at 20 μg/ml | 19.2 ± 0.4 | 59.1 ± 9.4 | 95.1 ± 11.5 | 57.3 ± 6.9 | 28.5 ± 3.5 | 6.6 ± 1.0 |
| Ethanol control | 20.3 ± 0.3 | 90.2 ± 3.1 | 101.1 ± 2.9 | 64.9 ± 2.4 | 25.7 ± 0.5 | 6.9 ± 0.1 |
| Water control | 30.1 ± 0.2 | 83.1 ± 17.7 | 100.1 ± 4.5 | 61.7 ± 2.4 | 26.9 ± 1.7 | 6.6 ± 0.5 |

### Example 3

### In Vitro Evaluation of a Food Supplement of the Invention Prepared from a Primary Product of Fermenting Monascus on Sterilized Wheat

1. Preparation of the Food Supplement

A food supplement of the invention is prepared from a product of fermenting Monascus on stone-ground wheat, passed through a sieve, then sterilized at 120° C. for 30 min. according to a protocol analogous to that used in Example 1.

The sterilization step eliminates the microbial strains that are initially present in the substrate that might interfere with the development of Monascus.

After cooling, the sterilized wheat is inoculated with wheat bran that has already been fermented (30° C. for 4 days) with the same Monascus species.

The material is homogenized in order to thoroughly distribute the Monascus spores in the entire mass of sterilized wheat.

The obtained mixture is then incubated under partially-anaerobic conditions at the temperature of 30° C. and in darkness for a period of 2 to 3 weeks. The fermentation medium is stirred daily during the first 3 days of incubation.

The mixture obtained at the end of the two- to three-week fermentation period can be used as-is as a food supplement, or it may be dried prior to use.

2. Evaluation of the Food Supplement

The in vitro effect of the food supplement obtained by culturing Monascus on metabolite product by methanogenic archaea was evaluated using a protocol analogous to that described in Example 2 (adding 100 μA of Monascus fermentation medium extract), except that the incubation time between the two transfers is 24 hrs. and not 48 hrs. By way of comparison, two control experiments were implemented:
Control Experiment 1: 100 μl of water was added in place of the Monascus fermentation medium extract
Control Experiment 2: 100 μl of a “wheat” substrate extract that was not in contact with Monascus in place of the Monascus fermentation medium extract

At the end of each of the 24-hr. incubation periods, gas production and methane concentrations were measured.

Strikingly, at +48 hrs. and +72 hrs, we observed a significant decrease in the quantities of methane produced by incubating the ruminal fluid in the presence of the food supplement of the invention as compared to the control experiments. More specifically, the quantities of methane produced in the presence of the food supplement of the invention were reduced by a factor of 2 and by a factor of 4, at +48 hrs. and at +72 hrs. respectively, as compared to the control experiments. Remarkably, no significant difference was observed in methane production between the two control experiments, which confirms that the decrease in methane quantities observed for ruminal fluid incubated in the presence of the food supplement results from the metabolites formed by Monascus from the wheat-based substrate.

### Example 4

### In Vivo Effects of a Food Supplement Composition on Metabolite Production by Ruminants

### A. Material and Methods

An in vivo trial on sheep was performed in order to validate the benefit of this concept. Over several weeks, six adult male Texel sheep were adapted to a maintenance diet composed of hay and rice (1:1). The animals, whose average body weight was 63.5±4 kg, received 1.2 kg of dry feed once a day, in the morning. Over 11 days, each animal received fermented rice produced in our laboratory, and was put back on its initial diet for two weeks.

Methane production was measured daily before and during treatment, but also 2 weeks after treatment. The concentration of volatile fatty acids in the ruminal contents was also analyzed using gas-phase chromatography. Tracking of methanogenic archaea and total bacteria was measured using quantitative PCR methods. Protozoans were counted using microscopy.

### B. Results

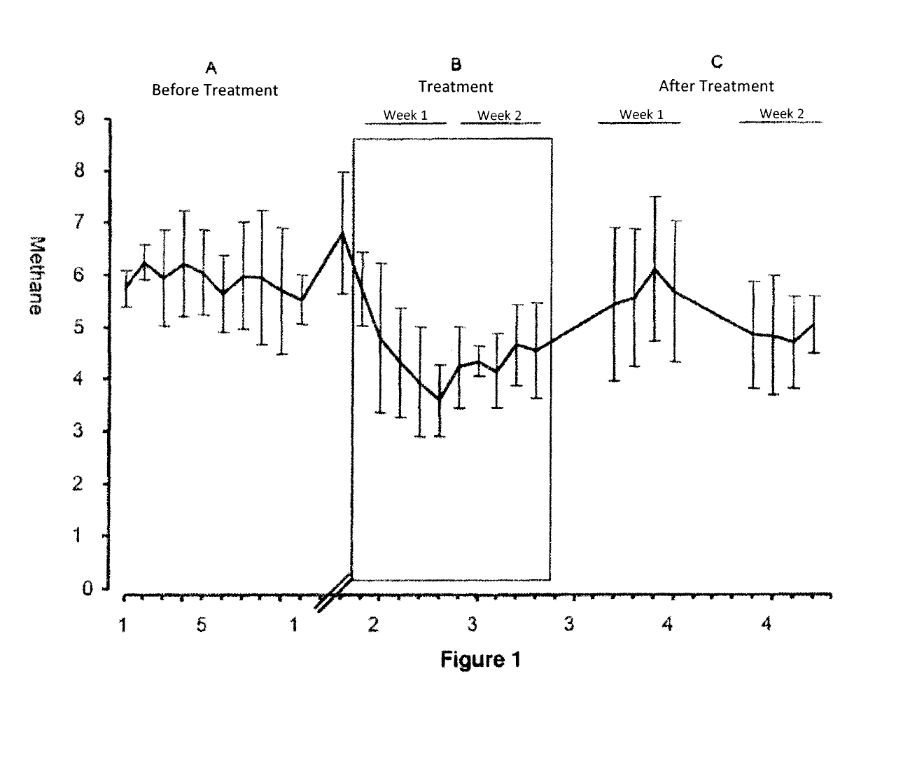

The results are presented in FIG. 1 and in Table 3 below.

Daily methane emissions decreased by an average of 30% during treatment (P<0.05) (Table 3 and FIG. 1). Fermentations shifted towards an increased proportion of propionate at the expense of acetate. In these animals, treatment led to a significant decrease in the number of methanogenic Archaea without changing the number of bacteria or protozoans in the rumen.

TABLE 3

| | Before | | | | | |
|---|---|---|---|---|---|---|
| | Treat- | Treatment | | Post-Treatment | | |
| | ment | w1 | w2 | w1 | W2 | SEM |
| Methane (L/day) | 59.3 a | 41.8 c | 43.4 C | 56.3 ab | 48.0 bc | 3.85 |
| Total VFA | 73.4 b | 61.8 c | 75.3 b | 94.4 a | 89.6 a | 3.84 |
| (μmol/L) | | | | | | |
| Acetate (A, %) | 70.7 b | 59.8 c | 63.6 fa | 66.0 a | 67.4 a | 3.84 |
| Propionate (P, %) | 12.6 b | 17.9 ab | 16.5 ab | 15.7 a | 15.6 ab | 2.26 |
| Butyrate (%) | 13.1 | 16.5 | 15.7 | 13.8 | 12.9 | 1.32 |
| Iso-acids (%) | 2.5 b | 3.1 b | 2.0 b | 3.0 a | 2.5 b | 0.27 |
| A:P | 5.8 a | 3.5 b | 4.0 b | 4.6 ab | 4.3 ab | 0.38 |

1.-13. (canceled)

14. A method for reducing methane production in ruminants, wherein said ruminants are supplied with an appropriate quantity of a food supplement composition comprising a product originating from fermentation of a substrate by at least one fungal microorganism belonging to the Monascus genus.

15. The method of claim 14, wherein the fungal microorganism is selected from the group consisting of Monascus albidulus, Monascus argentinensis, Monascus aurantiacus, Monascus barkeri, Monascus bisporus, Monascus eremophilus, Monascus floridanus, Monascus fuliginosus, Monascus fumeus, Monascus kaoliang, Monascus lunisporas, Monascus mucoroides, Monascus olei, Monascus pallens, Monascus paxii, Monascus pilosus, Monascus pubigerus, Monascus purpureus, Monascus ruber, Monascus rubropunctatus, Monascus rutilus, Monascus sanguineus, Monascus serorubescens, and Monascus vitreus.

16. The method of claim 14, wherein the fungal microorganism is a strain of Monascus ruber species.

17. The method of claim 14, wherein the substrate is selected from rice, bread, bran, grains, grain byproducts, grain-based substances, fodder, a nutrient medium for fungal microorganisms, or cellulose.

18. The method of claim 14, wherein the substrate is a solid substrate prepared from one or several products selected from the group composed of grains of the Triticum genus, grains of the Oriza genus, and products derived from said grains.

19. The method of claim 18, wherein the solid substrate is prepared from cereal grains to which wheat bran may optionally be added, in the absence of any additional nutrient compound.

20. The method of claim 14, wherein the substrate is obtained by steaming rice.

21. The method of claim 14, wherein the substrate is an extract of a product of fermentation by Monascus.

22. The method of claim 21, wherein said extract is an ethanolic extract.

23. A food supplement composition for reducing methane production in ruminants, comprising a product originating from fermentation of a substrate by at least one fungal microorganism belonging to the Monascus genus.

24. The composition of claim 23, wherein the fungal microorganism is selected from the group consisting of Monascus albidulus, Monascus argentinensis, Monascus aurantiacus, Monascus barkeri, Monascus bisporus, Monascus eremophilus, Monascus floridanus, Monascus fuliginosus, Monascus fumeus, Monascus kaoliang, Monascus lunisporas, Monascus mucoroides, Monascus olei, Monascus pallens, Monascus paxii, Monascus pilosus, Monascus pubigerus, Monascus purpureus, Monascus ruber, Monascus rubropunctatus, Monascus rutilus, Monascus sanguineus, Monascus serorubescens, and Monascus vitreus.

25. The composition of claim 23, wherein the fungal microorganism is a strain of Monascus ruber species.

26. The composition of claim 23, wherein the substrate is a solid substrate prepared from grains selected from the group composed of grains belonging to the Triticum genus, grains belonging to the Oriza genus, mixtures of said grains, and products derived from said grains.

27. The composition of claim 26, wherein the solid substrate is prepared from cereal grains, to which wheat bran may optionally be added, in the absence of any additional nutrient compound.
